(19) United States
(12) Patent Application Publication
(10) Pub. No.: US 2020/0253929 A1
(43) Pub. Date: Aug. 13, 2020

(54) TREATMENT OF EPILEPSY

(71) Applicants: KATHOLIEKE UNIVERSITEIT LEUVEN, Leuven (BE); THE UNIVERSITY COURT OF THE UNIVERSITY OF ABERDEEN, Aberdeen (GB)

(72) Inventors: Daniëlle Copmans, Holsbeek (BE); Peter De Witte, Kessel-Lo (BE); Rainer Ebel, Aberdeen (GB); Marcel Jaspars, Aberdeen (GB); Annelii Ny, Mol (BE); Mostafa Rateb, Glasgow (GB); Alan Smith, Aberdeen (GB); Jioji Tabudravu, Aberdeen (GB)

(21) Appl. No.: 16/642,492

(22) PCT Filed: Aug. 28, 2018
(86) PCT No.: PCT/EP2018/073159

§ 371 Date: Feb. 27, 2020

(30) Foreign Application Priority Data
Aug. 28, 2017 (GB) 1713763.9

(51) Int. Cl. A61K 31/407; A61K 36/062; A61P 25/08

(52) U.S. Cl. CPC A61K 31/407; A61P 25/08; A61K 36/062

(57) ABSTRACT

The present invention discloses pseurotins and azaspirofurans and their use in the treatment and prevention in epilepsy and other seizures. The present invention further discloses methods to screen pseurotin- and azaspirofuran-like molecules as pharmaceutically active compounds.

### BACKGROUND OF THE INVENTION

Epilepsy is one of the most common neurological conditions, affecting more than 70 million people of all ages with no geographical, social, or racial boundaries [Ngugi et al. (2010) Epilepsia 51, 883-890; Sander (2003) Curr Opin Neurol 16, 165-170. Singh and Trevick (2016) Neurol Clin 34, 837-847] Epilepsy is a disease of the brain that is characterized by spontaneous recurrent unprovoked seizures [Fisher (2015) Curr Opin Neurol 28, 130-135]. Despite an exponential growth of marketed antiseizure drugs (ASDs) over the past 25 years, seizures remain uncontrolled in one third of the patients due to drug resistance [Franco et al. (2016) Pharmacol Res 103, 95-104]. As uncontrolled epilepsy is associated with increased physical and physiological comorbidities and increased risk of sudden unexplained death, there is a substantial burden on the patients, caretakers and society [Golyala and Kwan (2017) Seizure 44, 147-156]. Hence, more efficacious ASDs that can treat patients with drug-resistant seizures are sorely needed [Bialer et al. (2017) Epilepsia 58, 181-221].

Zebrafish animal models for screening compounds for anti-epileptic activity have been described [Howe et al. (2013) Nature 496, 498-503; MacRae and Peterson (2015) Nat Rev Drug Discov 14, 721-731; Khan et al. (2017) Br J Pharmacol. 174, 1925-1944].

Pseurotins are a family of fungal secondary metabolites that exhibit a wide range of bioactivities [Tsunematsu et al. (2014) Angew Chem Int Ed Engl 53, 8475-8479], e.g., pseurotin A is known to inhibit monoamine oxidase [Maebayashi et al. (1985) JSM Mycotoxins 1985, 33-34], to induce adrenergic neuronal cell differentiation [Komagata et al. (1996), J Antibiot (Tokyo) 49, 958-959], to have apomorphine antagonistic activity [EP0546475], to exert antibacterial activity [Mehedi et al. (2010) Asian J. Chem. 22, 2611-2614], and to inhibit chitin synthase [Wenke et al. (1993) Biosci. Biotech. Biochem. 57, 961-964] and immunoglobuline E production [Ishikawa et al. (2009) Bioorg Med Chem Lett 19, 1457-1460]. Azaspirofurans are chemically very similar to pseurotins, but have an ethyl furan ring instead of a vicinal diol (FIG. 1). Little is known about the bioactivities of azaspirofurans as they were identified only recently [Ren et al. (2010) Arch. Pharm. Res. 33, 499-502]. So far, azaspirofuran A was reported to specifically inhibit the proliferation of the A459 cancer cell line [Ren et al. (2011) Chin. Pharm. J. 46, 569-575].

### SUMMARY OF THE INVENTION

The present invention discloses hetero-spirocyclic γ-lactams which were isolated from the bioactive marine fungus Aspergillus fumigatus. The compounds were investigated for antiseizure activity in the larval zebrafish PTZ seizure model. Pseurotin A₂ and azaspirofuran A were found to have antiseizure activity, which was not present to the same extent in the other chemical analogues tested and thus demonstrating a structure-specific interaction with their target(s). The antiseizure activity of pseurotin A₂ and azaspirofuran A translated to a mouse model of drug-resistant focal seizures and in addition, both compounds were found to be drug-like after ADMET analysis.

Reductions in PTZ-induced seizure behavior by pseurotin D, 11-anthranilyl-pseurotin A, compound code 1JB, pseurotin F1, and 11-O-methylpseurotin A (FIG. 2), which suggest at least some antiseizure activity. Besides, pseurotin A shows significant anti-epileptiform activity (FIG. 4H).

Based on the prominent antiseizure activity in zebrafish and mice and the drug-likeness, the present invention relates to pseurotin A₂ and azaspirofuran A as compounds in the use for the treatment of for the treatment of (epileptic) seizures and in the treatment of epilepsy in general

The present invention demonstrates antiseizure activity of certain pseurotins and azaspirofurans, and gives another example of the translation of results from zebrafish larvae to mice.

The present invention accordingly relates to the screening of other hetero-spirocyclic γ-lactams and modified versions thereof for compounds which are suitable for the prevention and treatment of seizures.

The invention is summarized in the following statements:

1. A pseurotin or an azaspirofura for use in the treatment or prevention of epilepsy. The pseurotin or azosipran for use in the treatment or prevention of epilepsy, selected from the group consisting of Pseurotin A, Pseurotin A₂ and Azaspirofuran A, pseurotin D, 11-anthranilyl-pseurotin A, compound code 1JB, pseurotin F1, and 11-O-methylpseurotin A and Pseurotin A.

2. A method for identifying a pharmaceutical compound against epilepsy, the method comprising the steps of: providing a compound comprising a moiety with chemical formula:

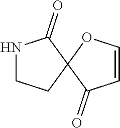 [SMILES: O=C1C=COC12CCNC2=O]

and testing the compound for antiseizure activity.

3. The method according to statement 2, wherein is a pseurotin or an azaspirofuran.

4. The method according to statement 2 or 3, wherein the pseurotin or azaspirofuran is a compound as depicted in FIG. 1, with modified molecular structure or stereochemistry.

5. The method according to any one of statements 2 to 4, wherein anti-seizure activity is determined in a zebrafish model.

6. The method according to any one of statements 2 to 5, wherein anti-seizure activity is further determined in a mammalian model.

7. The method according to any one of statements 2 to 6, further comprising the step of testing the compound for a side effect.

8. The method according to any one of statements 2 to 7, further comprising the step of formulating a compound with determined anti-seizure activity into a pharmaceutical composition with an acceptable carrier, for use in the treatment of epilepsy.

### DETAILED DESCRIPTION OF THE INVENTION

### BRIEF DESCRIPTION OF THE FIGURES

FIG. 1. Chemical structures isolated from extracts of the marine fungus Aspergillus fumigatus.

FIG. 2. Behavioral antiseizure analysis in the zebrafish PTZ seizure model. Screening for antiseizure activity of compounds at their maximum tolerated concentrations (MTC, Table 1) in the zebrafish pentylenetetrazole (PTZ) seizure model after 2 hours (A) and 18 hours (B) of incubation. PTZ-induced seizure-like behavior is expressed as mean actinteg units/5 min (±SD) during the 30 minutes recording period. Number of replicate wells per condition: 60 (A) and 82-83 (B) larvae were used for vehicle (VHC)+PTZ and VHC+VHC controls, 8-12 (A) and 9-14 (B) larvae were used for compound+PTZ conditions, and 4-12 (A) and 9-12 (B) larvae were used for compound+VHC conditions. Data are pooled from multiple experimental plates. Statistical analysis: one-way ANOVA with Dunnett's multiple comparison test for comparison of compound+PTZ conditions with VHC+PTZ controls and Kruskal-Wallis test with Dunn's multiple comparison test for comparison of compound+VHC conditions with VHC+VHC controls (GraphPad Prism 5). Significance levels: * p≤0.05; ** p≤0.01; *** p≤0.001.

FIG. 3. Behavioral antiseizure analysis of azaspirofuran A and pseurotin A₂ in the zebrafish PTZ seizure model. Antiseizure activity of azaspirofuran A (A-B) and pseurotin A₂ (C-D) in the zebrafish pentylenetetrazole (PTZ) seizure model after 2 hours (A-B) and 18 hours (C-D) of incubation, respectively. PTZ-induced seizure-like behavior is expressed as mean actinteg units/5 min (±SEM) during the 30 minutes recording period (A, C) and over 5 minute time intervals (B, D). Means are pooled from three independent experiments with each 9-12 replicate wells per condition. Statistical analysis: (A, C) one-way ANOVA with Dunnett's multiple comparison test, (B, D) two-way ANOVA with Bonferroni posttests (GraphPad Prism 5). Significance levels: * p≤0.05; ** p≤0.01; *** p≤0.001. Abbreviation: vehicle, VHC.

FIG. 4. Electrophysiological antiseizure analysis of azaspirofuran A, azaspirofuran B, pseurotin A₂ and pseurotin A in the zebrafish PTZ seizure model. Noninvasive local field potential recordings from the optic tectum of larvae pre-exposed to vehicle (VHC) and pentylenetetrazole (PTZ), VHC only, compound and PTZ, or compound and VHC. (A-D) Larvae were incubated with 12.5 μg/mL azaspirofuran (azasp.) A or B for 2 hours, (E-H) or with 12.5 μg/mL pseurotin A₂ or 100 μg/mL pseurotin A for 18 hours, conform with the optimal condition used in the behavioral assay. Epileptiform discharges are quantified by the number (mean±SD) (B, F) and cumulative duration (mean±SD) (C, G) of events per 10 minute recording. Larvae are considered to possess epileptiform brain activity when three or more events occurred during a 10 minute recording (A, E). (D, H) Mean duration (±SEM) of epileptiform events based on the mean durations of all larvae that demonstrated one or more events. Number of replicates per condition: 33 (A-C), 32 (D), 22 (E-G), and 20 (H) larvae were used for VHC+PTZ controls, 31 (A-C), 9 (D), 22 (E-G), and 10 (H) larvae were used for VHC+VHC controls, 15 (A-C), 9-12 (D), 13-19 (E-G), and 13 (H) larvae were used for compound+PTZ conditions, and 15 (A-C), 4-5 (D), 14-19 (E-G), and 2-6 (H) larvae were used for compound+VHC conditions. Statistical analysis: (A, E) Fisher's exact test with Bonferroni posttest, (B-D, F-H) Kruskal-Wallis test with Dunn's multiple comparison test (GraphPad Prism 5). Significance levels: * p≤0.05; ** p≤0.01; *** p≤0.001.

FIG. 5. Antiseizure activity analysis of azaspirofuran A and pseurotin A₂ in the mouse 6-Hz psychomotor seizure model. Drug-resistant psychomotor seizures were induced by electrical stimulation (6 Hz, 0.2 ms rectangular pulse width, 3 s duration, 44 mA) through the cornea, 30 minutes after i.p. injection of vehicle (VHC, n=10), positive control valproate (n=6), or test compound (n=6-7), and 120 minutes after i.p. injection of negative control phenytoin (n=5). Percentage of mice protected against seizures are depicted (A) and defined by a seizure duration shorter than 27 seconds (s) (B). Seizure durations (mean±SD) are depicted (B). Statistical analysis: (A) Fisher's exact test, (B) one-way ANOVA with Dunnett's multiple comparison test (GraphPad Prism 5). Significance levels: * p≤0.05; ** p≤0.01; *** p≤0.001.

### ABBREVIATIONS USED IN THE APPLICATION

ACSF, artificial cerebrospinal fluid; ASD, antiseizure drug; azasp., azaspirofuran; dpf, days post-fertilization; DMSO, dimethyl sulfoxide; FP7, Seventh Framework Programme; LFP, local field potential; min, minute; MTC, maximum tolerated concentration; PEG200, polyethylene glycol M.W. 200; PTZ, pentylenetetrazole; t_{1/2}, half-life; VHC, vehicle

Definitions

“Pseurotins” and “azaspirofurans” are structures with a hetero-spirocyclic γ-lactam substituted e.g. with a side chain with a vicinal diol or an ethyl furan group. Hydroxyl groups in a vicinal diol may be further substituted (such as methyl in 11-O-methylpseurotin A. Other related compounds which are envisaged in the screening methods include azaspirenes, synerazoles, cephalimysines berkeleyamides.

The above indicated medical use of the compounds equally comprises the use of the salt form thereof. Pharmaceutically acceptable salts include those described by Berge, Bighley and Monkhouse, J. Pharm. Sci., 1977, 66, 1-19.

Compounds are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The corresponding stereospecific name and structure have been assigned to the final product where the enantiomeric excess of said product is greater than 70%. Assignment of absolute stereochemistry is based on the known chirality of the starting material. In examples where the composition of the final product has not been characterized by chiral HPLC, the stereochemistry of the final product has not been indicated. However, the chirality of the main component of the product mixture will be expected to reflect that of the starting material and the enantiomeric excess will depend on the synthetic method used and is likely to be similar to that measured for an analogous example (where such an example exists). Thus compounds shown in one chiral form are expected to be able to be prepared in the alternative chiral form using the appropriate starting material. Alternatively, if racemic starting materials are used, it would be expected that a racemic product would be produced and the single enantiomers could be separated by the usual methods. The invention also extends to any tautomeric forms and mixtures thereof.

“Seizure” refers to a brief episode of signs or symptoms due to abnormal excessive or synchronous neuronal activity in the brain. The outward effect can vary from uncontrolled jerking movement (tonic-clonic seizure) to as subtle as a momentary loss of awareness (absence seizure).

Seizure types are typically classified on observation (clinical and EEG) rather than the underlying pathophysiology or anatomy.

I Focal seizures (Older term: partial seizures)

IA Simple partial seizures—consciousness is not impaired

IA1 With motor signs

IA2 With sensory symptoms

IA3 With autonomic symptoms or signs

IA4 With psychic symptoms

IB Complex partial seizures—consciousness is impaired (Older terms: temporal lobe or psychomotor seizures)

IB1 Simple partial onset, followed by impairment of consciousness

IB2 With impairment of consciousness at onset

IC Partial seizures evolving to secondarily generalized seizures

IC1 Simple partial seizures evolving to generalized seizures

IC2 Complex partial seizures evolving to generalized seizures

IC3 Simple partial seizures evolving to complex partial seizures evolving to generalized seizures

II Generalized seizures

IIA Absence seizures (Older term: petit mal)

IIA1 Typical absence seizures

IIA2 Atypical absence seizures

IIB Myoclonic seizures

IIC Clonic seizures

IID Tonic seizures,

IIE Tonic-clonic seizures (Older term: grand mal)

IIF Atonic seizures

III Unclassified epileptic seizures

A more recent classification is published in Fisher et al (2017) Epilepsia 58(4) 522-530.

“Epilepsy” is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

A large number of subtypes of epilepsy have been characterized and categorized. The classification and categorization system, that is widely accepted in the art, is that adopted by the International League Against Epilepsy's (“ILAE”) Commission on Classification and Terminology [See e.g., Berg et al., “Revised terminology and concepts for organization of seizures,” Epilepsia, 51(4):676-685 (2010)]:

I. Electrochemical syndromes (arranged by age of onset):
I.A. Neonatal period: Benign familial neonatal epilepsy (BFNE), Early myoclonic encephalopathy (EME); Ohtahara syndrome
I.B. Infancy: Epilepsy of infancy with migrating focal seizures; West syndrome; Myoclonic epilepsy in infancy (MEI); Benign infantile epilepsy; Benign familial infantile epilepsy; Dravet syndrome; Myoclonic encephalopathy in non-progressive disorders
I.C. Childhood: Febrile seizures plus (FS+) (can start in infancy); Panayiotopoulos syndrome; Epilepsy with myoclonic atonic (previously astatic) seizures; Benign epilepsy with centrotemporal spikes (BECTS); Autosomal-dominant nocturnal frontal lobe epilepsy (ADNFLE); Late onset childhood occipital epilepsy (Gastaut type); Epilepsy with myoclonic absences; Lennox-Gastaut syndrome; Epileptic encephalopathy with continuous spike-and-wave during sleep (CSWS), also known as Electrical Status Epilepticus during Slow Sleep (ESES); Landau-Kleffner syndrome (LKS); Childhood absence epilepsy (CAE)
I.D. Adolescence-Adult: Juvenile absence epilepsy (JAE);Juvenile myoclonic epilepsy (JME); Epilepsy with generalized tonic-clonic seizures alone; Progressive myoclonus epilepsies (PME); Autosomal dominant epilepsy with auditory features (ADEAF); Other familial temporal lobe epilepsies
I.E. Less specific age relationship: Familial focal epilepsy with variable foci (childhood to adult); Reflex epilepsies

II. Distinctive constellations
II.A. Mesial temporal lobe epilepsy with hippocampal sclerosis (MTLE with
II.B. Rasmussen syndrome
II.C. Gelastic seizures with hypothalamic hamartoma
II.D. Hemiconvulsion-hemiplegia-epilepsy
E. Epilepsies that do not fit into any of these diagnostic categories, distinguished on the basis of presumed cause (presence or absence of a known structural or metabolic condition) or on the basis of Primary mode of seizure onset (generalized vs. focal)

III. Epilepsies attributed to and organized by structural-metabolic causes
III. A. Malformations of cortical development (hemimegalencephaly, heterotopias, etc.)
III. B. Neurocutaneous syndromes (tuberous sclerosis complex, Sturge-Weber, etc.)
III. C. Tumor
III. D. Infection
III. E. Trauma

IV. Angioma
IV.A. Perinatal insults
IV.B. Stroke
IV.C. Other causes

V. Epilepsies of unknown cause
Vi. Conditions with epileptic seizures not traditionally diagnosed as forms of epilepsy per se
   VI.A. Benign neonatal seizures (BNS)
   VI.B. Febrile seizures (FS)

A more recent classification can be found in Scheffer et al. (2017) Epilepsia. 58, 512-521.

A first aspect of the present invention relates to a pseurotin or an azaspirofuran for use in the treatment or prevention of epilepsy, more particularly for preventing and alleviating seizures.

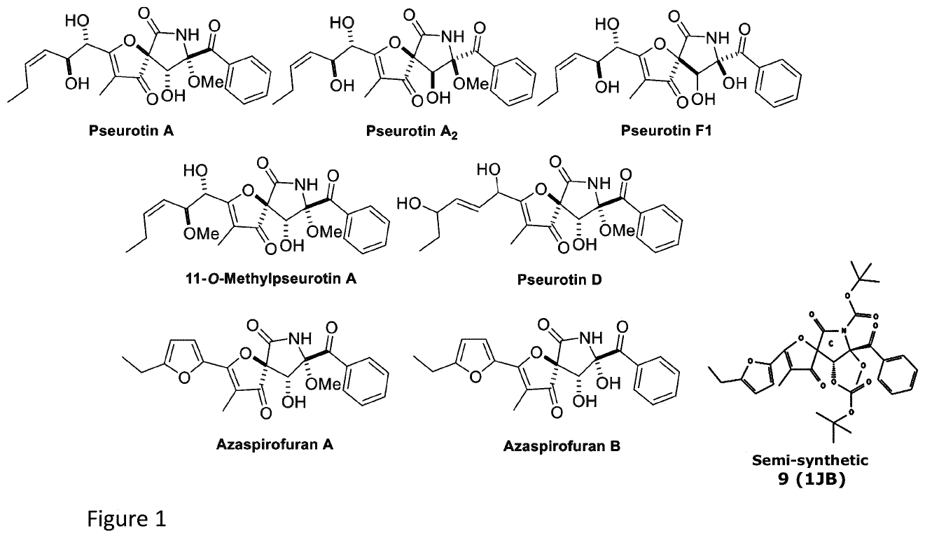

Preferred compounds are pseurotin A₂ and azaspirofuran A (both depicted in FIG. 1).

The examples of the present invention used specific compounds isolated from one Aspergillus strain. Herein some are effective in the zebrafish and mouse seizure models, while other show no pharmaceutical activity. The screening did not include all pseurotins and azaspirofuran, or other hetero-spirocyclic γ-lactams described in the literature, let alone chemically modified versions.

Thus another aspect of the invention relates to the testing of compounds for antiseizure activity, wherein the compounds comprise a moiety with the following chemical structure:

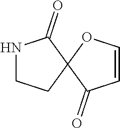 [SMILES: O=C1C=COC12CCNC2=O]

Natural, synthetic and semi-synthetic compounds comprising such moiety can be found in chemical databases such as Reacsys™.

The present invention relates in another aspect to methods to identify other hetero-spirocyclic γ-lactam, such as pseurotins and azaspirofurans in the zebrafish and mouse model to identify compounds with a similar or higher activity than pseurotin A₂ and azaspirofuran A, and or with better ADMET properties.

Candidate pseurotins and pseurotin like molecules described in the art are for example described in

WO2016198850: pseurotin B, pseurotin C, pseurotin E, pseurotin F2;

Saraiva et al. Nat Prod Res. (2015) 29(16), 1545-50: pseurotin FD-838;

Wakefield et al Front Microbiol. (2017) 8, 1284: Pseurotin G and 11-O-methylpseurotin A;

Lee et al. J Nat Prod. (2016) 79(12), 2983-2990: pseurotins A3 and G, along with the known compounds FR-111142, pseurotins A, A1, A2, D, and F2, 14-norpseurotin A;

Sbaraina BMC Genomics. (2016) 17(Suppl8):736: pseurotin-related compound (MaNRPS-PKS2);

Yamamoto et al Angew Chem Int Ed Engl. (2016) 55, 6207-6210. azaspirene and synerazol;

Watanabe Chem. Pharm. Bull. 62(12) 1153-1165 (2014): cephalimysin and compounds disclosed in Rateb et al. (2013) RSC Advances 3, 14444-14450; Wang et al. (2011) Can. J. Chem. 89, 72-76; EP0546474; Breitenstein et al. (1981) Helvetica Chim. Acta 64, 379-388; Tsunematsu et al. (2014) Angew Chem Int Ed Engl 53, 8475-8479; Maebayashi et al. (1985) JSM Mycotoxins 1985, 33-34 Komagata et al. (1996), J Antibiot (Tokyo) 49, 958-959, EP0546475, Mehedi et al. (2010) Asian J. Chem. 22, 2611-2614 and Wenke et al. (1993) Biosci. Biotech. Biochem. 57, 961-964.

Methods of the present invention for drug screening can be advantageously performed with a zebrafish model which is suitable for large-scale screening and captures the complexity of a whole body organism and the central nervous system. As a vertebrate, zebrafish are highly similar to humans due to a high genetic, physiological and pharmacological conservation [Howe et al. (2013) Nature 496, 498-503; MacRae and Peterson (2015) Nat Rev Drug Discov 14, 721-731; Khan et al. (2017) Br J Pharmacol. 174, 1925-1944]. Moreover, given the small size of embryos and larvae, they fit in the well of microtiter plates and hence are suitable for medium to high-throughput testing. Given the low volumes used in 96- and 384-well plates, zebrafish larvae only require small amounts of sample in the low microgram range when added to their swimming water and even less when administered by injection. This property is of particular interest for marine natural product drug discovery, where material is often scarce [West and Crawford (2016) Planta Med 82, 754-760]. A particular suitable larval zebrafish seizure and epilepsy model, is the larval zebrafish pentylenetetrazole (PTZ) seizure model. This model has the following advantages 1) the model has been extensively characterized in terms of behavioral and non-behavioral seizure markers, 2) it has been pharmacologically characterized with ASDs on the market, 3) results translate well to rodent models, 4) seizures can easily and rapidly be induced by a single administration of the convulsant drug to the larva's aqueous environment, and 5) seizures can be quantified automatically by video recording [Baraban et al. (2005) Neurosci 131, 759-768; Afrikanova et al. (2013) PLoS One 8, e54166; Berghmans et al. (2007) Epilepsy Res 75, 18-28; Buenafe et al. (2013) ACS Chem Neurosci 4, 1479-1487; Orellana-Paucar. et al. (2012) Epilepsy Behav 24, 14-22].

Within the framework of PharmaSea, 5 pseurotins (pseurotin A, pseurotin A₂, pseurotin Fl, 11-O-methylpseurotin A, and pseurotin D) and 2 azaspirofurans (azaspirofuran A and B) were isolated from extracts of the bioactive marine fungus Aspergillus fumigatus, which was collected from a Red Sea sediment in Hurghada, Egypt. All compounds were investigated for antiseizure activity in the larval zebrafish PTZ seizure model, after acute and chronic exposure. Despite close structural similarities, only pseurotin A₂ and azaspirofuran A significantly ameliorated PTZ-induced seizures, which is indicative of a highly specific interaction. In addition electrophysiological analysis from the zebrafish midbrain demonstrated that pseurotin A₂ and azaspirofuran A also significantly lowered PTZ-induced epileptiform discharges, thereby demonstrating anti-epileptiform activity. Methods of the present invention for drug screening can be equally performed in a mammalian model, wherein generally those compounds are tested which already gave positive results in the above mentioned zebrafish model.

Accordingly, pseurotin A₂ and azaspirofuran A were tested in the mouse 6-Hz (44 mA) psychomotor seizure model.

Herein, treatment with pseurotin A₂, as well as with azaspirofuran A shortened the seizure duration concentration-dependently. These results not only confirmed the translation of results from zebrafish larvae to mice but also suggest that pseurotin A₂ and azaspirofuran A are effective against drug-resistant focal seizures.

Methods of the present invention for drug screening can further comprise the step of determining parameters such as absorption, distribution, metabolism, and excretion—toxicity.

ADMET profiles are elucidated for pseurotin A₂ and azaspirofuran A and showed that both compounds are drug-like.

In summary, based on the prominent antiseizure activity seen in a standard zebrafish and mouse seizure model and their ADMET characteristics, the present invention claims pseurotin A₂ and azaspirofuran A as compounds for use in the treatment of seizures.

### EXAMPLES

### Example 1

### Isolation and Structural Elucidation of Compounds from the Bioactive Marine Fungus Aspergillus Fumigatus

The marine fungal isolate MR2012 used in this study was isolated from a Red Sea sediment in Hurghada, Egypt, in the context of the PharmaSea project and taxonomically identified on a molecular basis as Aspergillus fumigatus [EI-Gendy and Rateb (2015) Bioorg. Med. Chem. Lett. 25, 3125-3128].

Compounds pseurotin A, pseurotin A₂, pseurotin F1, 11-O-methylpseurotin A, pseurotin D, and azaspirofurans A and B, all known hetero-spirocyclic γ-lactams, were isolated from the CH₂Cl₂ fraction of the fungal fermentation (FIG. 1). These compounds are disclosed in Rateb et al. (2013) RSC Advances 3, 14444-14450; Wang et al. (2011) Can. J. Chem. 89, 72-76; EP0546474; Breitenstein et al. (1981) Helvetica Chim. Acta 64, 379-388 and Ren et al. (2010) Arch. Pharm. Res. 33, 499-502. Their structures were confirmed by HRESIMS analysis and by comparing the 1D and 2D NMR and optical rotation with literature data as indicated. In addition, two compounds with structural analogy were identified: 11-O-methylpseurotin A and 11-anthranilyl-Pseurotin A.

### Example 2

### Azaspirofuran A and Pseurotin A₂ Ameliorate Seizures in the Zebrafish PTZ Seizure Model

To investigate whether the isolated compounds display antiseizure activity, they were tested in the zebrafish PTZ seizure model after both an acute (2 hours) and chronic (18 hours) incubation time at their maximum tolerated concentration (MTC) (Table 1, FIG. 1).

TABLE 1
Maximum tolerated concentrations (MTCs) of tested pseurotins
and azaspirofurans

| Compound | MTC (μg/mL) |
|---|---|
| pseurotin A | 100 |
| pseurotin A₂ | 12.5 |
| pseurotin F1 | 50 |
| 11-O-methylpseurotin A | 100 |
| pseurotin D | 100 |
| 11-anthranilyl-Pseurotin A | 100 |
| 1JB | 100 |
| azaspirofuran A | 12.5 |
| azaspirofuran B | 12.5 |

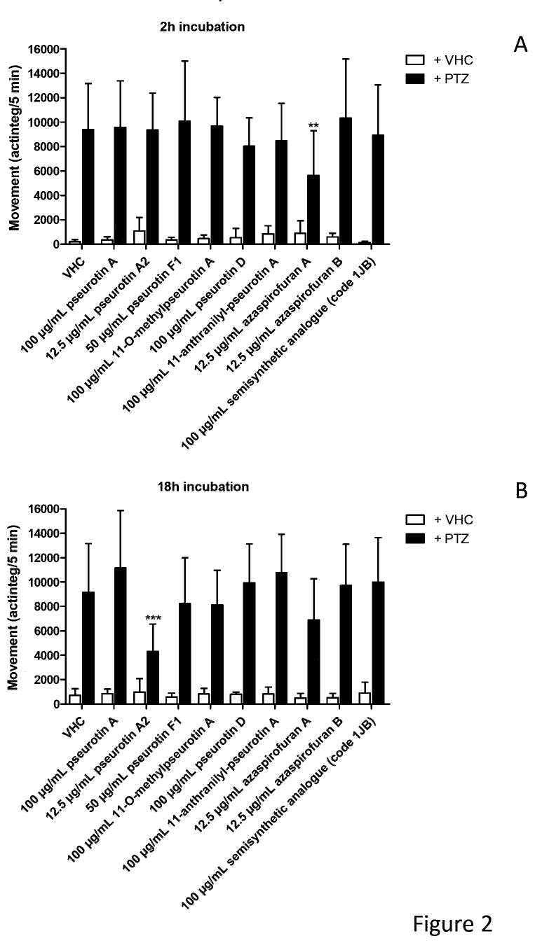

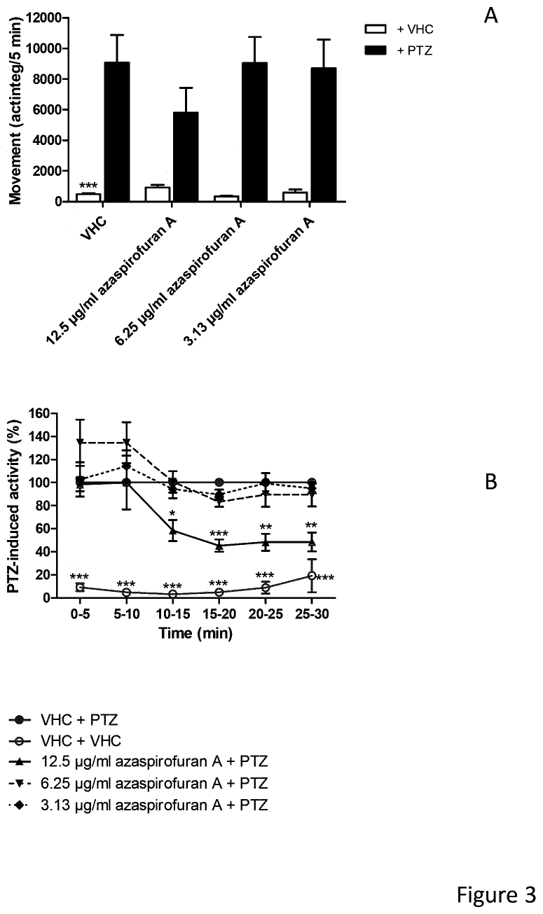

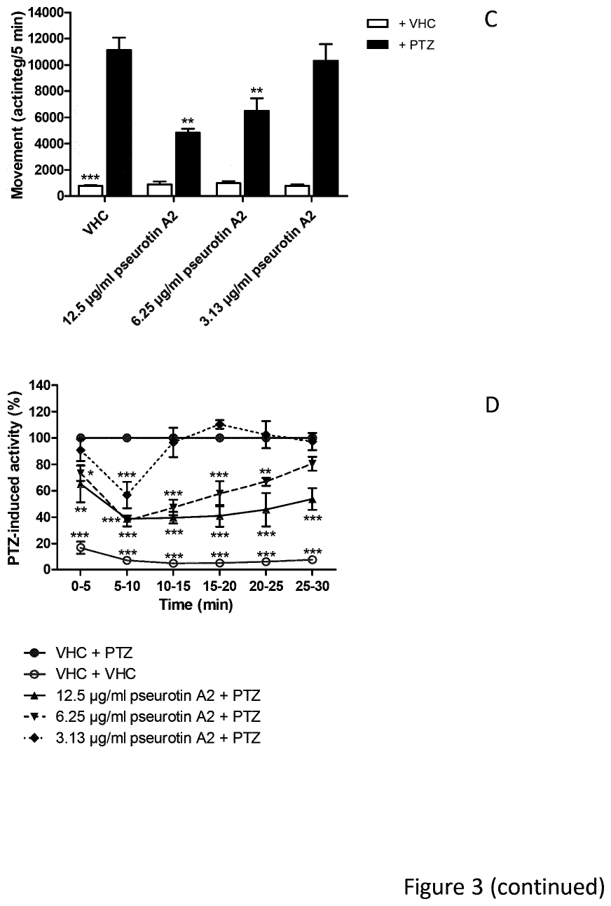

The MTC was defined as the highest concentration at which no larvae died nor showed signs of toxicity or locomotor impairment in comparison to vehicle (VHC)-treated control larvae. In case no MTC was reached, 100 μg/mL was used as the test concentration. In line with studies previously reported [Baraban et al. (2005) Neurosci 131, 759-768; Afrikanova et al. (2013) PLoS One 8, e54166], addition of the GABA_{A}-receptor antagonist PTZ to the swimming water of 7 days post-fertilization (dpf) zebrafish larvae strongly elevated larval locomotion (p≤0.001) (FIGS. 2 and 3) as a result of induced seizures (or seizure-like behavior) that were recognized as typical high-speed swimming, whirlpool-like circling, clonus-like seizures, and loss of posture, as previously described in Baraban et al cited above. Azaspirofuran A (p≤0.01) and pseurotin A₂ (p≤0.001) significantly lowered PTZ-induced seizures of larvae after 2 and 18 hours of compound exposure, respectively (FIG. 2). In addition, pseurotin D, 11-anthranilyl-pseurotin A, compound code 1JB, pseurotin F1, and 11-O-methylpseurotin A. showed a less pronounce reduction in PTZ-induced seizures, thereby suggesting antiseizure activity.

These results suggest that azaspirofuran A and pseurotin A₂ specifically interact with their antiseizure target(s). It is unlikely that the activity of azaspirofuran A is due to an improved compound uptake as the Log P of azaspirofuran A and B is predicted to be 3.98 and 3.81, respectively, and good compound bioavailability in zebrafish larvae is expected from a Log P of 1 onwards [Milan et al. (2003) Circulation 107, 1355-1358]. The close analogues pseurotin A and A₂ are diastereomers with different configurations at C-8 and C-9 (FIG. 1) [Wang et al. (2011) Can. J. Chem. 89, 72-76]. Their structural differences do not affect uptake, which is also expected to be good with a Log P of 3.23.

So, likely the structural differences result in distinct pharmacological activities rather than in altered bioavailabilities. Nevertheless the pharmacokinetics of the individual compounds in zebrafish larvae are currently unknown, so it cannot be ruled out that the actual brain concentrations of azaspirofuran A and pseurotin A₂ are higher than those of their inactive analogues. Of note, like azaspirofuran A, pseurotin A, pseurotin D, and 11-Omethylpseurotin A also possess the methoxyl group and are not active, which can be due to the absence of the ethyl furan ring. These observations suggest that the molecular target(s) of azaspirofuran A and pseurotin A₂ are not necessarily the same. Interestingly, antiseizure activity has not yet been reported for azaspirofurans or pseurotins.

To investigate the concentration-response relationship, azaspirofuran A and pseurotin A₂ were retested at their MTC, MTC/2, and MTC/4 (two-fold serial dilution) in the zebrafish PTZ seizure model at their optimal incubation time (2 and hours, respectively) in three independent experiments (FIG. 3). Azaspirofuran A lowered PTZ-induced seizure behavior to the same extent as before at the MTC over the 30 minute (min) recording period (FIG. 3A). A more detailed analysis of the 30 min recording period into 5 min time intervals revealed a significant reduction in the 10-30 min time window (p≤0.001, p≤0.01 and p≤0.05, FIG. 3B). No antiseizure activity was seen at lower concentrations. Pseurotin A₂ showed concentration-dependent activity against PTZ-induced seizure behavior, both within the 30 min recording period (p≤0.01) (FIG. 3C) as during the 5 min time intervals (p≤0.001, p≤0.01 and p≤0.05, FIG. 3D).

### Example 3

### Azaspirofuran A and Pseurotin A₂ Ameliorate Epileptiform Brain Activity in the Zebrafish PTZ Seizure Model

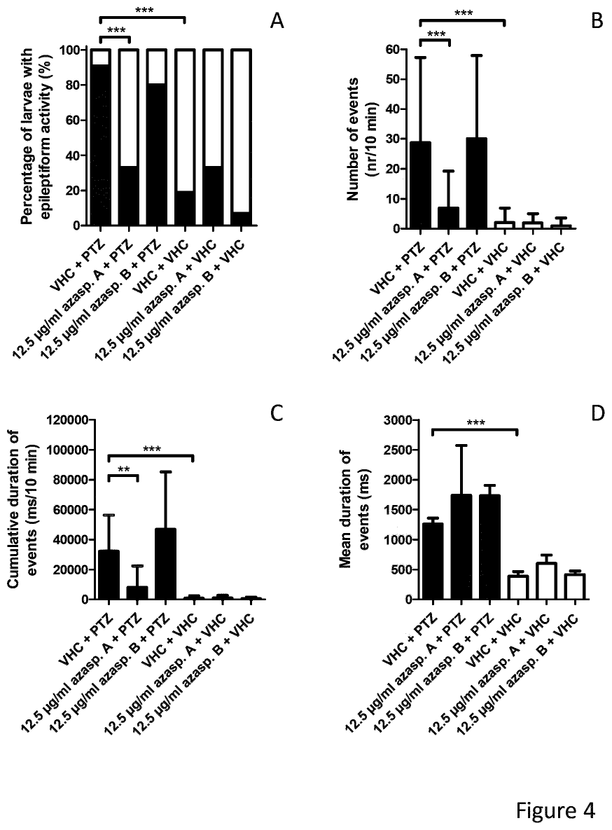

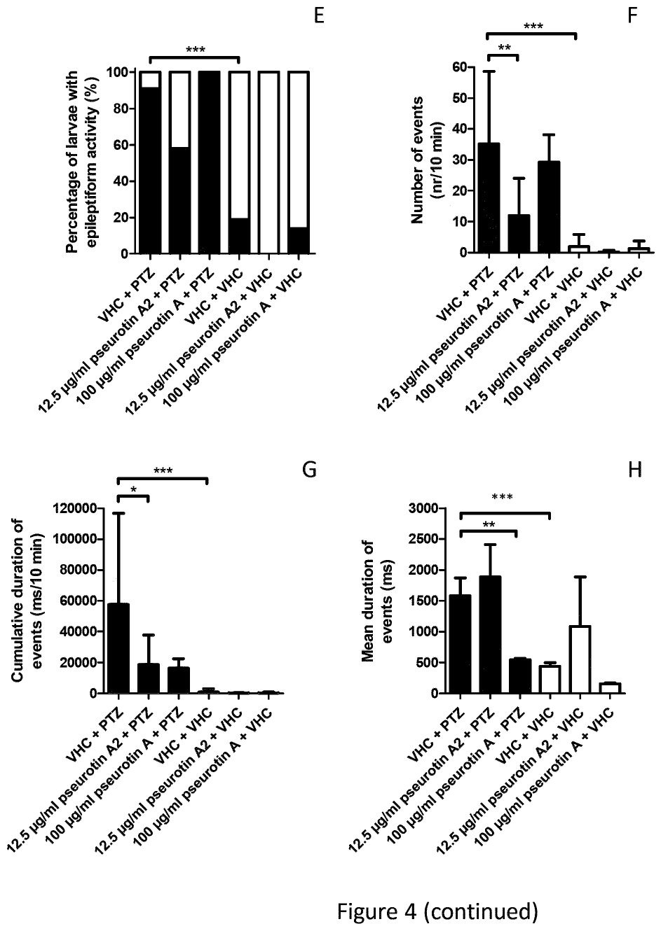

To determine whether azaspirofuran A and pseurotin A₂ can ameliorate the PTZ-induced hyperexcitable state of the brain that is characterized by epileptiform discharges, local field potential (LFP) recordings were non-invasively measured from the midbrain (optic tectum) of zebrafish larvae [Zdebik et al. (2013) PLoS One 8, e79765; Britton et al. (2016) In Electroencephalography (EEG): An Introductory Text and Atlas of Normal and Abnormal Findings in Adults, Children, and Infants (St. Louis and Frey, Eds.), Chicago]. To that end larvae were treated with either VHC or test compound (MTC and optimal incubation time were used) followed by a 15 min during exposure to PTZ or VHC prior to the electrophysiology measurements (FIG. 4). Pre-incubation with azaspirofuran A significantly reduced (p≤0.001) the percentage of larvae with PTZ-induced epileptiform activity with almost 60% in comparison to controls (VHC+PTZ) (FIG. 4A). A larva was considered to have epileptiform brain activity when at least 3 electrical discharges were seen in the 10 min recording period that fulfilled the pre-defined requirements of an epileptiform event (see methods). Larvae also showed significantly less epileptiform events (p≤0.001) when pre-exposed to azaspirofuran A, in comparison to controls (VHC+PTZ), resulting in a shorter cumulative duration of events (p≤0.01) over a 10 min recording period (FIG. 4B and C). Pseurotin A₂ only non-significantly lowered the percentage of larvae with epileptiform activity with 33%, in comparison to controls (VHC+PTZ) (FIG. 4E). However, larvae did show significantly less epileptiform events (p≤0.01) in comparison to controls (VHC+PTZ), resulting in a shorter cumulative duration of events (p≤0.05) over a 10 min recording period (FIGS. 4F and G). Thus, both compounds not only ameliorate PTZ-induced seizures but are likely to do so by lowering the PTZ-induced hyperexcitable state of the brain. Of note, the structural analogue pseurotin A was observed to non-significantly lower the number and cumulative duration of epileptiform events in comparison to controls (FIG. 4F and G), but also to significantly lower the mean duration of epileptiform events (p≤0.01, FIG. 4H). Hence, azaspirofuran A, pseurotin A₂ and pseurotin A demonstrate anti-epileptiform activity.

### Example 4

### Azaspirofuran A and Pseurotin A₂ Ameliorate Focal Seizures in the Mouse 6-Hz (44 mA) Psychomotor Seizure Model

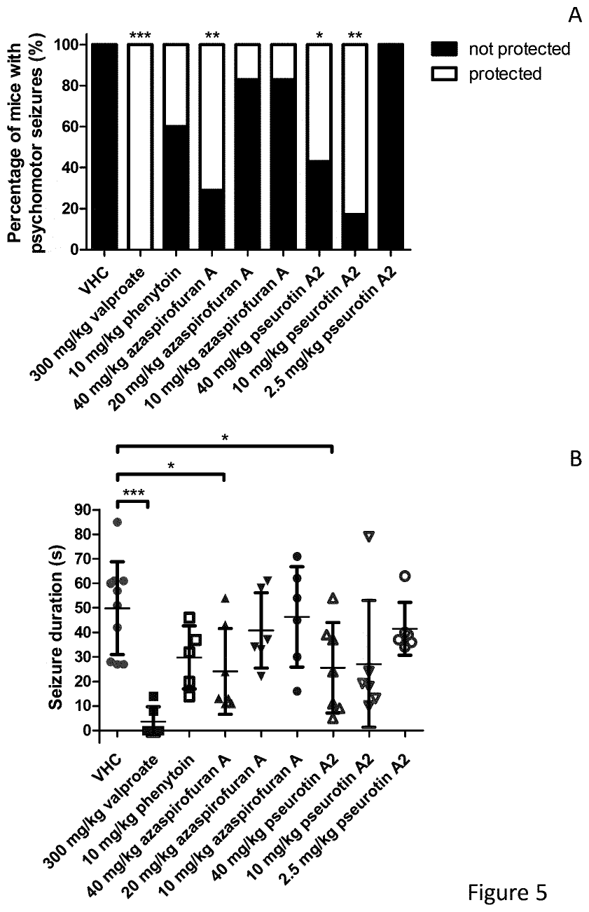

Although the zebrafish model has a high degree of genetic, physiological and pharmacological conservation¹³, it is more distinct from humans than mammals. Therefore, we were interested to see if the observed antiseizure properties of azaspirofuran A and pseurotin A₂ would translate to a rodent model. The mouse 6-Hz (44 mA) psychomotor seizure model was chosen, because it can detect compounds with novel antiseizure mechanisms and with potential against drug-resistant seizures [Barton et al. (2001) Epilepsy Res 47, 217-227; Wilcox et al. (2013) Epilepsia 54 S4, 24-34]. In this model drug-resistant focal impaired awareness seizures [Fisher et al. (2017) Epilepsia 58, 531-542], previously referred to as complex partial or psychomotor seizures [Holcomb and Dean (2011) Psychomotor Seizures, In Encyclopedia of Child Behavior and Development (Goldstein, S., and Naglieri, J. A., Eds.), pp 1191-1192, Springer US, Boston, Mass.], are induced by low frequency, long duration corneal electrical stimulation (6 Hz, 0.2 ms rectangular pulse width, 3 s duration, 44 mA) and are characterized by stun, twitching of the vibrissae, forelimb clonus, and Straub tail. Mice injected i.p. with VHC (PEG200:DMSO:water; 0.25:0.25:0.5, 30 min before electrical stimulation) had a mean (±SD) seizure duration of 50 seconds (±19 seconds) with a minimum of 27 seconds. Stimulated mice with a seizure duration shorter than 27 seconds were thus considered to be protected. In line with previous studies [Orellana-Paucar et al. (2013) PLoS One 8, e81634 Barton et al. cited above], i.p. administration of the positive control valproate (300 mg/kg dose, 30 min before electrical stimulation) protected all mice against the induced seizures (p≤0.001) with a mean seizure duration of 4 seconds (±6 seconds). In contrast, seizures showed only a limited non-significant response to the negative control phenytoin (10 mg/kg dose, i.p. injected 120 min before electrical stimulation, as reported by Barton et al. cited above and Walrave et al. (2015) Epilepsy Res 115, 67-72; Leclercq et al. (2015) Epilepsy Behav 45, 53-63. Administration of 40 mg/kg azaspirofuran A (i.p. injected, 30 min before electrical stimulation) protected 5 out of 7 mice against induced seizures (p≤0.01) and lowered seizure duration to a mean of 24 seconds (±18 seconds) (p≤0.05) in comparison to VHC-controls. Administration of 40 mg/kg pseurotin A₂ (i.p. injected, 30 min before electrical stimulation) protected 4 out of 7 mice against induced seizures (p≤0.05) and lowered seizure duration to a mean of 26 seconds (±18 seconds) (p≤0.05) in comparison to VHC-controls. While azaspirofuran A showed a trend for dose-dependent activity with no significant effects seen at 20 and 10 mg/kg, pseurotin A₂ showed dose-dependent activity as it was also effective at 10 mg/kg, protecting 5 out of 6 mice (p≤0.01) with a mean seizure duration of 27 seconds (±26 seconds) and was inactive at 2.5 mg/kg (FIG. 5). These data confirm the antiseizure properties of azaspirofuran A and pseurotin A₂ in a standard mouse model of drug-resistant focal seizures. The identification and validation of these novel antiseizure hits thereby demonstrate the effectiveness of using the larval zebrafish model for ASD discovery. Besides, this study provides another example of the translation of results from zebrafish to rodent seizure models.

Example 5

### ADMET Profiling of Azaspirofuran A and Pseurotin A₂

Finally, to define the drug-likeness of the antiseizure compounds, the ADMET profiles of azaspirofuran A and pseurotin A₂ were elucidated by the research institute (MEDINA, Granada, Spain) using standard in vitro assays. The ADMET results are summarized in Table 2.

TABLE 2
ADMET Analysis of Azaspirofuran A and Pseurotin A₂

| ADMET test | Azaspirofuran A | | Pseurotin A₂ | |
|---|---|---|---|---|
| | Cytotoxicity | | | |
| HEPG2 cells | IC₅₀ > 50 μM | No effect | IC₅₀ > 50 μM | No effect |
| THLE2 cells | IC₅₀ > 50 μM | Weak | IC₅₀ > 50 μM | Weak |
| | | decrease | | decrease |
| SHSY5Y | IC₅₀ > 50 μM | Weak | IC₅₀ = 17.9 μM | Decrease |
| cells | | decrease | | |
| | Cardiotoxicity | | | |
| Naᵥ1.5 | IC₅₀ = 39.06 μM | Low | IC₅₀ > 50 μM | No effect |
| channel | | inhibitory | | |
| | | effect | | |
| Caᵥ1.2 | IC₅₀ > 50 μM | No effect | IC₅₀ > 50 μM | No effect |
| channel | | | | |
| hERG | IC₅₀ > 50 μM | No effect | IC₅₀ > 50 μM | No effect |
| channel | | | | |
| | CYP450 enzymes | | | |
| CYP3A4 | IC₅₀ > 88 μM | No effect | IC₅₀ > 88 μM | No effect |
| CYP2D6 | IC₅₀ = 23.4 μM | Weak | IC₅₀ > 88 μM | No effect |
| | | inhibition | | |
| CYP2C9 | IC₅₀ = 47.3 μM | Weak | IC₅₀ > 88 μM | No effect |
| | | inhibition | | |
| | Other factors | | | |
| Hepatic | 20.17 μL/ | Medium | <8.6 μL/ | Low |
| clearance | min/mg | | min/mg | |
| | protein (t_{1/2} = | | protein (t_{1/2} > | |
| | 33.12 minutes) | | 60 minutes) | |
| Kinetic | >100 μM | Acceptable | >100 μM | Acceptable |
| solubility | | | | |
| Protein | 95.42% | Low | 36.53% | High |
| binding | | percentage | | percentage |
| | | of free | | of free |
| | | drug; | | drug; |
| | | 66.7% | | 100% |
| | | recovery | | recovery |
| Permeability | 18.64 × 10⁻⁶ | High | 0.05 × 10⁻⁶ | Low |
| | cm s⁻¹ | | cm s⁻¹ | |

Abbreviation: concentration at which an assay is inhibited by 50%, IC₅₀.

No notable cytotoxicity or cardiotoxicity was observed for either one of the compounds. Both compounds showed an acceptable solubility and azaspirofuran A also demonstrated a high permeability, a desired combination that is not common. Furthermore, azaspirofuran A only weakly inhibited the CYP2D6 and CYP2C9 enzymes and did not affect the CYP3A4 enzyme, while pseurotin A₂ did not inhibit any of the three types of CYP450 enzymes. Thus, azaspirofuran A and pseurotin A₂ are unlikely to present drug-drug interactions. In addition, they are metabolically stable with a half-life (t_{1/2}) of 33 and >60 minutes for azaspirofuran A and pseurotin A₂, respectively. Finally, azaspirofuran A shows a high level of plasma protein binding (95%) with low recovery, which is not optimal but can be addressed, and pseurotin A₂ has a much lower plasma protein binding of only 37% with good recovery. Thus, azaspirofuran A and pseurotin A2 show very promising ADMET characteristics and are therefore considered to be drug-like. Hence, we disclose azaspirofuran A and pseurotin A₂ as compounds for the treatment of seizures in general and drug-resistant focal seizures in specific.

### Example 6

### Methods

6.1. Chemical Experimental Procedures

NMR data were acquired on a Varian VNMRS 600 MHz NMR spectrometer. High resolution mass spectrometric data were obtained using a Thermo LTQ Orbitrap coupled to an HPLC system (PDA detector, PDA autosampler, and pump). The following conditions were used: capillary voltage of 45 V, capillary temperature of 260° C., auxiliary gas flow rate of 10-20 arbitrary units, sheath gas flow rate of 40-50 arbitrary units, spray voltage of 4.5 kV, and mass range of 100-2000 amu (maximal resolution of 30000). For LC/MS, a C18 analytical HPLC column (5 82 m, 4.6 mm×150 mm) was used with a mobile phase of 0 to 100% MeOH over 30 min at a flow rate of 1 mL min⁻¹. Biotage Flash system SP1-XOB1, Charlottesville, Wash., USA. Compound purification was conducted using Agilent 1200 HPLC system with a Waters Sunfire C18 column (5 82 m, 100 Å, 10 mm×250 mm), connected to a binary pump, and monitored using a photodiode array detector.

6.2. Microbial Strain

The marine fungal isolate MR2012 used in this study was isolated from a Red Sea sediment in Hurghada, Egypt in September 2011 in the framework of the PharmaSea project, and taxonomically identified on a molecular basis as Aspergillus fumigatus²⁰.

6.3. Microbial Fermentation, Extraction, and Isolation

The fungal isolate MR2012 initially cultured on a solid medium composed of (g/L) glucose 10, yeast extract 10, malt extract 4. A 6 liter fermentation was conducted on a medium composed of (g/L) sucrose 100, glucose 10, casamino acids 0.1, yeast extract 5, MOPS 21, K₂SO₄ 0.25×10−6, MgCl₂.6H₂O 1.0×10⁻⁶ for 12 days at 30° C. with shaking at 180 rpm. At the end of the incubation period, Diaion HP-20 resin was added to the culture media and shaken for 6 hours at 180 rpm, then cultures were centrifuged (3000 rpm for 20 min) where the residue composed of the fungal mycelia and resin were washed with distilled water twice and extracted with MeOH, and subjected to LC-HRESIMS analysis. This extract was fractionated successively with n-hexane (3×250 mL), CH₂Cl₂ (3×300 mL), and then EtOAc (3×250 mL). Each solvent fraction was evaporated in vacuo and subjected to LC-HRESIMS and ¹H NMR analysis, which revealed that the CH₂Cl₂ fraction was the one of interest to follow. This CH₂Cl₂ fraction was loaded on Flash Biotage using a FLASH 65i cartridge, solvent methanol/water 0-100%, flow rate 60 mL/min over 20 min and UV collection wavelengths 225 and 254 nm to produce 6 fractions. All of these fractions were monitored by LC-HRESIMS. Injection of fraction 4 into Agilent HPLC system using semi-preparative Sunfire C18 column (250×10 mm, 5 82 m) with CH3CN:H2O 30-90% over 30 min with a 2 mL/min flow led to the isolation of 38 mg of pseurotin A, 30 mg of pseurotin A₂, 2 mg of pseurotin F1, 6 mg of 11-O-methylpseurotin A, and 5 mg of pseurotin D. Injection of fraction 5 into Agilent HPLC system using semi-preparative Sunfire C18 column (250×10 mm, 5 82 m) with CH₃CN:H₂O 40-80% over 30 min with a 2 mL/min flow led to the isolation of 26 mg of azaspirofuran A and 23 mg of azaspirofuran B.

6.4. Compound Preparation

For experiments with zebrafish larvae, dry samples were dissolved in 100% dimethyl sulfoxide (DMSO, spectroscopy grade) as 100-fold concentrated stocks and diluted in embryo medium to a final concentration of 1% DMSO content. Control groups were treated with 1% DMSO (VHC) in accordance with the final solvent concentration of tested compounds. For mice experiments, a mixture of poly-ethylene glycol M.W. 200 (PEG200), 100% DMSO (spectroscopy grade), and demineralized water (PEG200:DMSO:water; 0.25:0.25:0.5) was used as solvent and VHC.

6.5. Compound Log P Calculation

Log P (ACD/Log P) values were obtained from ChemSpider and predicted by means of the ACD/Labs Percepta Platform (PhysChem Module) based on the compound structure [Petrauskas and Kolovanov (2000) Persp. Drug Discov. Des. 19, 99-116].

6.6. Experimental Animals

All animal experiments carried out were approved by the Ethics Committee of the University of Leuven (approval numbers 101/2010, 061/2013, 150/2015, and 023/2017) and by the Belgian Federal Department of Public Health, Food Safety & Environment (approval number LA1210199).

6.6.1 Zebrafish

Adult zebrafish (Danio rerio) stocks of AB strain (Zebrafish International Resource Center, Oregon, USA) were maintained at 28.0° C., on a 14/10 hour light/dark cycle under standard aquaculture conditions. Fertilized eggs were collected via natural spawning and raised in embryo medium (1.5 mM HEPES, pH 7.2, 17.4 mM NaCl, 0.21 mM KCl, 0.12 mM MgSO₄, 0.18 mM Ca(NO3)₂, and 0.6 μM methylene blue) at 28.0° C., under constant light.

6.6.2. Mice

Male NMRI mice (weight 18-20 g) were acquired from Charles River Laboratories and housed in poly-acrylic cages under a 14/10-hour light/dark cycle at 21° C. The animals were fed a pellet diet and water ad libitum, and were allowed to acclimate for one week before experimental procedures were conducted. Prior to the experiment, mice were isolated in a poly-acrylic cage with a pellet diet and water ad libitum for habituation overnight in the experimental room, to minimize stress.

6.7 Zebrafish Pentylenetetrazole Seizure Model

6.7.1 Toxicity Evaluation

Maximum tolerated concentration (MTC) was determined prior to further experiments and used as the highest test concentration. MTC was investigated by exposing 12 larvae of 6 days post-fertilization (dpf) to a range of concentrations in a 100 μL volume during 18 hours. The following parameters were investigated after 2 and 18 hours of exposure: touch response, morphology, posture, edema, signs of necrosis, swim bladder, and heartbeat. MTC was defined as the highest concentration at which no larvae died nor showed signs of toxicity or locomotor impairment in comparison to VHC-treated control larvae. In case MTC was not reached, 100 μg/mL was used as the highest test concentration.

6.7.2. Behavioral Analysis

Experimental procedure was described in Afrikanova et al.(2013) PLoS One 8, e54166 and Orellana-Paucar et al. (2012) Epilepsy Behav 24, 14-22. In brief, a single 7 dpf larva (in case of 2 hours incubation) or 6 dpf larva (in case of 18 hours incubation) was placed in each well of a 96-well plate and treated with either VHC (1% DMSO) or compound (1% DMSO) in a 100 μL volume. Larvae were incubated in dark for 2 or 18 hours at 28° C., whereafter 100 μL of either VHC (embryo medium) or 40 mM PTZ (dissolved in embryo medium, 20 mM working concentration) was added to each well. Next, within 5 min the 96-well plate was placed in an automated tracking device (ZebraBox Viewpoint, France) and larval behavior was video recorded for 30 min. The complete procedure was performed in dark conditions using infrared light. Total locomotor activity was recorded by ZebraLab software (Viewpoint, France) and expressed in actinteg units, which is the sum of pixel changes detected during the defined time interval (5 min). Larval behavior was depicted as mean actinteg units/5 min during the 30 min recording period and over 5 min time intervals. Data are expressed as mean±SD or as mean ±SEM when the means of independent experiments were pooled.

6.7.3. Electrophysiology

Experimental procedure was described before [Zdebik et al. (2013) PLoS One 8, e79765; Sourbron et al. (2017) Front. Pharmac. 8, 191; Copmans et al. (2018) Neurochem. Int. 112, 124-133.]. Non-invasive LFP recordings were measured from the midbrain (optic tectum) of 7 dpf zebrafish larvae pre-incubated with VHC only, PTZ only, compound and VHC, or compound and PTZ. Larvae were incubated for approximately 2 or 18 hours with VHC (1% DMSO) or compound (1% DMSO) in a 100 μL volume. After incubation, 100 μL of VHC (embryo medium) or 40 mM PTZ (dissolved in embryo medium, 20 mM working concentration) was added to the well for 15 min prior to recording. These steps occurred at 28° C., while further manipulation and electrophysiological recordings occurred at room temperature (21° C.). The larva was embedded in 2% low melting point agarose (Invitrogen) and the signal electrode (an electrode inside a soda-glass pipet (1412227, Hilgenberg) pulled with a DMZ Universal Puller (Zeitz, Germany), diameter±20 microns, containing artificial cerebrospinal fluid (ACSF: 124 mM NaCl, 10 mM glucose, 2 mM KCl, 2 mM MgSO₄, 2 mM CaCl₂, 1.25 mM KH₂PO₄, and 26 mM NaHCO₃, 300-310 mOsmols)) was positioned on the skin covering the optic tectum. A differential extracellular amplifier (DAGAN, USA) amplified the voltage difference between the signal (measured by the signal electrode) and the reference electrode. The differential signal was band pass filtered at 0.3-300 Hz and digitized at 2 kHz via a PCI-6251 interface (National instruments, UK) using WinEDR (John Dempster, University of Strathclyde, UK). A grounding electrode grounded the electrical system. All electrodes were connected with ACSF. Each recording lasted 600 seconds. Manual analysis was completed by quantification of the number, cumulative duration, and mean duration of epileptiform-like events with Clampfit 10.2 software (Molecular Devices Corporation, USA). An electrical discharge was considered epileptiform if it was a poly-spiking event comprising at least 3 spikes with a minimum amplitude of three times the baseline amplitude and a duration of at least 100 milliseconds. Data are expressed as mean±SD for the number and cumulative duration of epileptiform events and as mean±SEM for the mean duration of events.

6.8. Mouse 6-Hz Psychomotor Seizure Model

Experimental procedure was described in Copmans et al. (2018) Neurochem. Int. 112, 124-133. before Buenafe et al. cited above; Orellana-Paucar cited above. In brief, NMRI mice (average weight 28 g, range 23-32 g) were randomly divided into control and treatment groups (n=5-10). 50 μL (injection volume was adjusted to the individual weight) of VHC (PEG200:DMSO:water; 0.25:0.25:0.5) or treatment (an ASD or test compound dissolved in VHC) was i.p. injected in mice and after 30 or 120 (in case of phenytoin, as reported by Barton et al. cited above) min psychomotor seizures were induced by low frequency, long duration corneal electrical stimulation (6 Hz, 0.2 ms rectangular pulse width, 3 s duration, 44 mA) using an ECT Unit 5780 (Ugo Basile, Comerio, Italy). Mice were manually restrained and a drop of ocular anesthetic (0.5% lidocaine) was applied to the corneas before stimulation. Following electrical current stimulation, the mouse was released in a transparent cage for behavioral observation, which was video-recorded. VHC-treated mice typically displayed stun, twitching of the vibrissae, forelimb clonus, and Straub tail for at least 27 seconds. In addition, facial and mouth jerking as well as head nodding were observed occasionally. Mice displaying normal exploratory and locomotion behavior within a time period of 26 seconds were considered to be protected against the induced psychomotor seizures. Seizure durations were measured during the experiment by experienced researchers, familiar with the different seizure behaviors. In addition, seizure durations were determined by blinded video analysis to confirm or correct the initial observations. Data are expressed as mean±SD.

6.9. ADMET Profiling

6.9.1. Cell Viability and MTT Assays

Protocol was based on literature [Patel and Patel. (2011) J. App. Pharm. Sci 1, 167-171]. Three cell lines were used: a) Hep G2 (HB-8065), a well-differentiated human hepatocellular carcinoma cell line, b) THLE-2, human liver epithelial cells transformed with SV40 large T-antigen and c) SHSYSY, a thrice-cloned sub-line of a human metastatic bone tumor. Cells were seeded at a concentration of 1×10⁴ cells/well in 200 μL culture medium and incubated at 37° C. in 5% CO₂ using 96-well plates for 24 h. Next, the medium was replaced with medium complemented with test compounds. After another 24 h incubation, the medium was replaced by 100 μL of a MTT (3-(4,5-dimethylthiazol-2-yl)-2,5-diphenyltetrazolium bromide) solution (5 mg/mL in PBS and diluted at 0.5 mg/mL in MEM without phenol red). The plates were gently shaken and incubated for 3 hours at 37° C. in a 5% CO₂ incubator. The supernatant was removed and 100 μL of 100% DMSO was added. The plates were gently shaken to solubilize the formed formazan. The absorbance was measured using a multireader (Victor2 (Wallac)) at a wavelength of 570 nm.

6.9.2. Cardiotoxicity

Fluorescence-based assays were performed using HEK293 cell lines that stably express the Naᵥ1.5-channel, Caᵥ1.2-channel or hERG K+-channel, using FMP Red Dye (Molecular Devices), calcium-sensitive fluorescent dye Fluo-4 (Invitrogen) and FIuxOR™ reagent (Invitrogen), respectively, and a FLIPR Tetra High-Throughput Cellular Screening System (Molecular Devices), according to manufacturer's protocols (Molecular Devices). Tetrodotoxin (TTX), israpidine and nicarpidine, and astemizole and haloperidol were used as standard sodium channel, calcium channel, and hERG channel blockers. Data were analyzed using Genedata Screener.

6.9.3 CYP450 Enzyme Inhibition Assay

Protocol was based on literature [Perez-Del Palacio et al. (2017) Front Pharmacol 8, 202]. Assessment of CYP450 inhibition was conducted in 96-well plate format at 37° C. Test compounds were dissolved in DMSO/acetonitrile (2 μL) and diluted in 98 μL NADPH solution (2 mM). Reactions were started by addition of 100 μL potassium phosphate buffer (200 mM, pH 7.4) containing Human Liver Microsomes (HLM) (0.5 mg/mL). Probe reactions for CYP3A4, CYP2D6 and CYP2C9 were conducted with 50 μM testosterone, 22 μM dextromethorphan and 10 μM diclofenac for 15 min. Reactions were terminated with the addition of a quench solution (90 μL) of acetonitrile containing internal standards for LC-MS/MS determination (60 ppb cortisone, 100 ppb 4′-hydroxydiclofenac 13C6, 60 ppb levallorphan).

6.9.4 Metabolic Stability Assay

The assay was performed with a mixture of test compounds (1 μM), NADPH (4 mM) and Human Liver Microsomes (HLM) (1 mg/mL) incubated at 37° C. Reactions were quenched at 0, 15, 30, 45, 60, and 90 min, using an equal volume of acetonitrile and then diluted 1:1 with water prior to analysis by LC-MS/MS. The analysis was performed using an Agilent Series 1290 LC system (Agilent Technologies, Santa Clara, Calif., USA) using a Supelco Discovery HS C18 (2.1×50 mm) 3 82 m column that was held at 30° C. Solvent A contained water with 0.1% formic acid and solvent B contained acetonitrile with 0.1% formic acid, and the flow rate was set at 400 μL/min. The gradient elution was performed as follows: 0-0.5 min 0% eluent B; 0.5-7 min 100% eluent B; 7-9 min 100% eluent B; 9-9.2 min 0% eluent B; and 9.2-10.5 min 0% eluent B. An API 4000 mass spectrometer in positive ESI mode (AB SCIEX, Concord, ON, Canada) was used with a generic method for data acquisition on all compounds. Data processing was performed using MultiQuant Software (AB SCIEX, Concord, ON, Canada) to process the data. Peak areas were used to plot the Ln % remaining relative to time (t)=0. The slope of the natural log of the percent remaining versus time was calculated to determine the first-order rate constant (k) and the half-life (t_{1/2}) of the test compounds according to the following equation: t_{1/2}=0.693/k (min)

6.9.5. Kinetic Solubility Assay

Protocol was based on literature [Perez et al. (2015) J Biomol Screen 20, 254-264]. The kinetic solubility assay was conducted in 96-well, flat-bottom, transparent polystyrene plates (Costar 9018, Corning, Tewksbury, Mass.). Six two-fold serial dilutions of an initial 10 mM test compound solution were prepared in DMSO. After a 2 h incubation period (to avoid missing slow precipitation) absorbance was measured at 620 nm by an EnVision multilabel plate reader. The kinetic solubility was estimated from the concentration of test compound that produced an increase in absorbance above the background levels (i.e. 1% DMSO in buffer).

6.9.6. Plasma Protein Binding Assay

Protocol was based on literature [Kumar Singh et al. (2012)1 Bioeq. Bioay. S14]. Rapid equilibrium dialysis was performed with RED device inserts (Thermo Scientific, Meridian Rd., Rockford, Ill.) containing dialysis membrane with a molecular weight cut-off of 8,000 Daltons. Serum (200 μL) containing test compound (5 μM) was added to the serum chamber of the insert and 350 μL of buffer was added to the buffer chamber of the insert. Dialysis was done at 37° C. with shaking at 100 rpm for 5 h. Following dialysis, an aliquot of 50 μL was removed from each well (plasma and buffer side) and diluted with an equal volume of opposite matrix to nullify the matrix effect. Then a fraction (50 μL) of each dialyzed sample was crashed with 150 μL of acetonitrile containing internal standard and vortexed for 1 min. The samples were centrifuged at 13300 rpm at 4° C. for 12 min and 100 μL of supernatant was used for LC-MS/MS analysis.

6.9.7. Parallel Artificial Membrane Permeability Assay (PAMPA)

The Gentest Pre-coated PAMPA Plate System (Corning) was used to perform the permeability assays. The 96 well filter plate, pre-coated with lipids, was used as the permeation acceptor and a matching 96 well receiver plate was used as the permeation donor. Compound solutions were prepared by diluting 10 mM DMSO stock solutions in PBS with a final concentration at 10 μM. The compound solutions were added to the wells (300 μL/well) of the receiver plate and PBS was added to the wells (200 μL/well) of the pre-coated filter plate. The filter plate was then coupled with the receiver plate and the plate assembly was incubated at RT without agitation for 5 h. Next, the plates were separated and 50 μL solution from each well of both the filter plate and the receiver plate was transferred to a vial with 150 μL acetonitrile and centrifuged at 13300 rpm for 10 min at 4° C. The supernatant was diluted in a solution water/acetonitrile (50/50). The final concentration of compounds in both donor wells and acceptor wells was analyzed by LC-MS/MS. Permeability of the compounds was calculated using the following equation:

Permeability (cm/s): Pe={−ln[1−CA(t)/Ceq]}/[A*(1/VD+1/VA)*t]}

A=filter area (0.3 cm2), VD=donor well volume (0.3 mL), VA=acceptor well volume (0.2 mL), t=incubation time (s), CA(t)=compound concentration in acceptor well at time t, CD(t)=compound concentration in donor well at time t, and Ceq=[CD(t)*VD+CA(t)*VA]/(VD+VA)]

1.-8. (canceled)

9. A method of treating epilepsy in a patient in need thereof, the method comprising administering an effective amount of a pseurotin or an azaspirofuran to the patient.

10. The method of claim 9, wherein the pseurotin or the azaspirofuran is selected from the group consisting of pseurotin D, 11-anthranilyl-pseurotin A, compound code 1JB, pseurotin F1, and 11-O-methylpseurotin A.

11. A method for identifying a pharmaceutical compound against epilepsy, the method comprising:
providing a compound comprising a moiety with chemical formula: 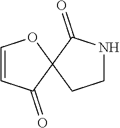 [SMILES: O=C1C=COC12CCNC2=O]
and,
testing the compound for antiseizure activity.

12. The method according to claim 11, wherein the compound is a pseurotin or an azaspirofuran.

13. The method according to claim 11, wherein the anti-seizure activity is determined in a zebrafish model.

14. The method according to claim 13, wherein the anti-seizure activity is further determined in a mammalian model.

15. The method according to claim 11, further comprising testing the compound for a side effect.

16. The method according to claim 11, further comprising formulating a compound with determined anti-seizure activity into a pharmaceutical composition with an acceptable carrier.
